(19) United States
(12) Patent Application Publication
(10) Pub. No.: US 2026/0027170 A1
(43) Pub. Date: Jan. 29, 2026

(54) BETA-Glucan-rich Saccharomyces Cerevisiae Yeast Cell Wall Extract in the Prevention or Treatment of a Disease Associated with or Caused by a Lawsonia Intracellularis Infection

(71) Applicant: Lesaffre et Compagnie, Paris (FR)

(72) Inventors: Julie SCHULTHESS, Lille (FR); Géraldine KUHN, La Madeline (FR)

(21) Appl. No.: 18/996,253

(22) PCT Filed: Jan. 13, 2023
(86) PCT No.: PCT/FR2023/051091

§ 371 Date: Jan. 17, 2025

(30) Foreign Application Priority Data
Jul. 20, 2022 (FR) FR2207447

(51) Int. Cl. A61K 36/064; A23K 10/16; A23K 20/163; A23K 50/30; A61K 31/716; A61P 31/04

(52) U.S. Cl. CPC A61K 36/064; A23K 10/16; A23K 20/163; A23K 50/30; A61K 31/716; A61P 31/04

(57) ABSTRACT

The invention relates to a β-glucan-rich Saccharomyces cerevisiae yeast cell wall extract for use in the prevention or treatment of a disease associated with or caused by a Lawsonia intracellularis infection in pigs, characterised in that the extract comprises β-glucans and mannans, where the β-glucans are present in the form of a mixture of β-1,3-glucans and β-1,6-glucans in a total amount of greater than or equal to 50% by weight, notably of between 50% and 99% by weight, and where the mannans are present in a total amount less than 5% by weight, notably of between 1% and 5% by weight.

### FIELD OF THE INVENTION

The present invention relates to the field of health, in particular animal health. The invention relates more particularly to a β-glucan-rich Saccharomyces cerevisiae yeast cell wall extract, and use thereof in the prevention or treatment of a disease associated with or caused by a Lawsonia intracellularis infection in pigs.

### TECHNICAL BACKGROUND

Lawsonia intracellularis, an obligate gram-negative intracellular bacterium, is the aetiological agent of porcine ileitis. Ileitis, also known as proliferative enteropathy, is one of the most common digestive diseases affecting grow-finish pigs worldwide.

The characteristic pathological feature of the disease is the proliferation of immature epithelial cells in the crypts of the ileum (the terminal part of the small intestine), the large intestine or both. Sections of infected tissue are characterised by a reddish thickening of the mucosa, resembling a ‘hosepipe’, and by enteric lesions. The thickening of the intestine eventually prevents normal intestinal function, hindering absorption capabilities and the transfer of nutrients. The clinical effects of the disease include chronic weight loss, lack of appetite, diarrhoea, and eventual death.

The disease may manifest in acute or chronic form. In the chronic form, which mainly affects pigs aged from 6 to 20 weeks, the clinical signs appear fairly mundane: reduced growth performance, i.e. decrease in daily weight gains, either with or without diarrhoea. As a result, the disease can often go undetected. The acute form, characterised by bloody diarrhoea and sudden death, affects pigs aged between 4 and 12 months. In infected herds. the disease is responsible for significant economic losses for livestock farmers, due to lower performance in terms of technical output, as well as higher medication costs and induced mortality.

At present, various vaccination strategies are available to combat ileitis. They generally involve parenteral administration (i.e. by injection) of the vaccine to young piglets. However, this approach is not entirely satisfactory given that it is relatively onerous for the livestock farmer.

There is therefore a real need to develop new methods for prevention and/or treatment of diseases associated with or caused by a Lawsonia intracellularis infection, such as ileitis in animals, in particular in pigs.

### SUMMARY OF THE INVENTION

The inventors herein have surprisingly found that β-glucan-rich Saccharomyces cerevisiae yeast cell wall extracts significantly boost the immunity of pigs that have been exposed to Lawsonia intracellularis, and thus provide the means for preventing and/or treating infections by the said bacterium, and/or diseases associated with or caused by the said bacterium, in particular ileitis.

More particularly, the inventors were able to demonstrate that the treatment of pigs exposed to Lawsonia intracellularis with β-glucan-rich Saccharomyces cerevisiae yeast cell wall extracts according to the invention made it possible both to promote weight gain in the pigs and to reduce the incidence of infection within the herd.

In addition, unlike parenteral vaccination strategies, the β-glucan-rich Saccharomyces cerevisiae yeast cell wall extract may advantageously be administered by the oral route, in particular in the feed, and can therefore act more directly in the intestine, where the pathogen is primarily present.

Accordingly, the object of the present invention relates to a β-glucan-rich Saccharomyces cerevisiae yeast cell wall extract for use in the prevention or treatment of a disease associated with or caused by a Lawsonia intracellularis infection in pigs, characterised in that the extract comprises β-glucans and mannans,
wherein the β-glucans are present in the form of a mixture of β-1,3-glucans and β-1,6-glucans in a total amount greater than or equal to 50% by weight, notably between 50% and 99% by weight; and
wherein the mannans are present in a total amount less than 5% by weight, notably between 1% and 5% by weight.

In certain embodiments, the disease is an inflammatory intestinal disease, in particular ileitis.

In certain embodiments, the total amount of β-glucans present in a β-glucan-rich Saccharomyces cerevisiae yeast cell wall extract is between 50% and 90% of the weight of the extract, for example between 50% and 80%, or between 50% and 70%, or indeed between 50% and 60%, of the weight of the extract.

In certain embodiments, the total amount of mannans present in a β-glucan-rich Saccharomyces cerevisiae yeast cell wall extract is between 2% and 4% of the weight of the extract.

In certain embodiments, the amount of mannans in the β-glucan-rich Saccharomyces cerevisiae yeast cell wall extract is such that the ratio of β-glucans/mannans is between 12 and 40 (weight/weight).

In certain embodiments, the amount of mannans in the β-glucan-rich Saccharomyces cerevisiae yeast cell wall extract is such that the ratio of β-glucans/mannans is between 15 and 30 (weight/weight), in particular between 18 and 22 (weight/weight).

In certain embodiments, the β-glucan-rich Saccharomyces cerevisiae yeast cell wall extract is characterised in that it has a dry matter content greater than or equal to 94% by weight, in particular greater than or equal to 96% by weight.

In certain embodiments, the β-glucan-rich Saccharomyces cerevisiae yeast cell wall extract is characterised in that it further comprises a protein content less than or equal to 10% by weight, and a glycogen content less than or equal to 10% by weight.

In certain embodiments, the β-glucan-rich Saccharomyces cerevisiae yeast cell wall extract is characterised in that the β-glucans and mannans are the only active ingredients of the β-glucan-rich Saccharomyces cerevisiae yeast cell wall extract.

In certain embodiments, the β-glucan-rich Saccharomyces cerevisiae yeast cell wall extract is characterised in that it is indeed Safglucan® which comprises β-glucans and mannans, wherein the β-glucans are present in the form of a mixture of β-1,3-glucans and β-1,6-glucans in a total amount of between 52 and 60% by weight; and the mannans are present in a total amount of between 1 and 5% by weight; and wherein the Safglucan® further comprises a protein content less than or equal to 10% by weight, and a glycogen content less than or equal to 10% by weight.

According to a second aspect, the invention relates to a composition comprising a β-glucan-rich Saccharomyces cerevisiae yeast cell wall extract and at least one bioactive agent for use in the prevention of an infection associated with the bacterium Lawsonia intracellularis in pigs, characterised in that the extract is as defined in the present patent description.

In certain embodiments, the bioactive agent is selected from the group consisting of antibiotics, vaccines, probiotics, prebiotics, vitamins, minerals, trace elements, and mixtures thereof.

According to a third aspect, the object of the invention relates to a pharmaceutical composition comprising a β-glucan-rich Saccharomyces cerevisiae yeast cell wall extract and at least one physiologically acceptable excipient for use in the prevention or treatment of an infection associated with the bacterium Lawsonia intracellularis in pigs, characterised in that the extract is as defined in the present description.

According to a fourth aspect, the invention relates to a feed for pigs comprising a β-glucan-rich Saccharomyces cerevisiae yeast cell wall extract for use in the prevention of a disease associated with or caused by a Lawsonia intracellularis infection in pigs, characterised in that the extract is as defined in the present description, or is part of a composition as defined in the present description.

### BRIEF DESCRIPTION OF THE FIGURES

FIG. 1 represents the weight of the animals during the test period.

FIG. 2 represents the average weight gain over the test period: black=control group, grey=treated group. **p<0.005, ***p<0.0001

FIG. 3 represents the quantification of the presence of Lawsonia intracellularis in the feces of the 2 groups of pigs (control: black histogram, treated: grey histogram). N=40 per group. P<0.0005 ***

### DETAILED DESCRIPTION

The invention is described in greater detail and in a non-limiting manner in the description that follows.

Unless stated otherwise, all percentages relating to quantities are percentages by weight.

As mentioned above, the present invention relates to β-glucan-rich Saccharomyces cerevisiae yeast cell wall extracts for use in the prevention or treatment of a disease associated with or caused by a Lawsonia intracellularis infection in animals, in particular pigs, characterised in that said extracts comprise β-glucans and mannans, wherein the β-glucans are present in the form of a mixture of β-1,3-glucans and β-1,6-glucans in a total amount greater than or equal to 50% by weight, notably between 50% and 99% by weight; and
wherein the mannans are present in a total amount less than 5% by weight, notably between 1% and 5% by weight.

In certain particular embodiments, the animal to which the invention applies is an animal selected from among farm animals and pets, in particular pig, or horses.

Accordingly, all the extracts, compositions, pharmaceutical compositions, and feedstuffs, which constitute the subject matter of the present invention and which are described hereinafter with reference to pigs, are also applicable to other animal species, in particular to horses.

As used herein, the term ‘pig’ refers to the porcine animal species in general.

As used here, the term ‘horse’ refers to the equidae family in general.

The term ‘disease associated with or caused by a Lawsonia intracellularis infection’ refers to diseases linked indirectly (‘associated’) or directly (‘caused’) to Lawsonia intracellularis infection.

### I—β-Glucan-Rich Saccharomyces cerevisiae Yeast Cell Wall Extracts

The β-glucans of an extract according to the invention are obtained from a Saccharomyces cerevisiae yeast, and more particularly from a Saccharomyces cerevisiae yeast strain. The state of the art includes numerous known strains of Saccharomyces cerevisiae. They are widely used in the agri-food industry for their role in the manufacture of a number of food products, most notably breads and fermented beverages.

As used herein, the term ‘yeast strain’ refers to a relatively homogeneous population of yeast cells obtained by culturing (or multiplying) the starter strain. A yeast strain is obtained from a clone, a clone being a population of yeast cells obtained from one single yeast cell. A Saccharomyces cerevisiae yeast strain may be cultured by using any appropriate method. Yeast culture methods are known in the prior art, and the person skilled in the art knows how to optimise the culture conditions for each strain based on its nature. Thus, Saccharomyces cerevisiae yeast may be obtained by culturing a strain in a suitable culture medium, for example, as described in the reference book ‘Yeast Technology’, 2nd edition, 1991, G. Reed and T. W. Nagodawithana, published by Van Nostrand Reinhold, ISBN 0-442-31892-8.

The terms ‘yeast cell walls’ and ‘yeast hulls’ are used interchangeably herein and refer to the insoluble fraction of yeast cells, i.e. the wall and the plasma membrane of the yeast. In a conventional manner, yeast cell walls are obtained by a method that comprises a step of autolysis or enzymatic hydrolysis, essentially by proteases, followed by a separation step for separating the soluble fraction from the insoluble fraction, with the isolated insoluble fraction corresponding to the yeast cell walls. The insoluble fraction can then be dried. The preparation method for obtaining yeast cell walls is such that it retains the structural polysaccharides of the cell wall, that is to say the β-glucans and the mannans, these mannans being in the form of mannoproteins. The preparation methods for obtaining yeast cell walls are known in the art (see for example the reference book “Yeast Technology”, 2nd edition, 1991, G. Reed and T. W. Nogodawithana, published by Van Nostrand Reinhold, New York, ISBN 0-442-31892-8).

A β-glucan-rich Saccharomyces cerevisiae yeast cell wall extract according to the invention refers to a fraction that has been extracted (or isolated) from the cell walls and which primarily contains β-glucans. The β-glucans derived from yeast cell walls are referred to as “cell wall β-glucans”. They are essentially glucose polymers whereof the glucose units in the main chain are linked by β-1,3 bonds, and whereof the branches are linked by β-1,6 bonds. Yeast β-glucans are insoluble and have low viscosity. The person skilled in the art knows how to extract β-glucans from yeast cell walls. A common method includes successive hot extractions with a base and with an acid (such as acetic acid), followed by aqueous washes in order to remove any soluble compounds from the cell walls, and recovery of the insoluble material consisting of cell wall β-glucans.

A β-glucan-rich Saccharomyces cerevisiae yeast cell wall extract according to the present invention comprises a total amount greater than or equal to 50% by weight of β-glucans in the form of a mixture of β-1,3-glucans and β-1,6-glucans. As used herein, the term “total amount greater than or equal to 50% by weight of β-glucans” indicates that the β-glucans represent at least half of the weight of the extract according to the invention. Thus, the total amount of β-glucans present in an extract according to the invention may represent between 50% and 90% of the weight of the extract, for example between 50% and 80%, or between 50% and 70%, or indeed between 50% and 60% of the weight of the extract.

In certain particular embodiments, the total amount of β-glucans present in an extract according to the invention represents between 50% and 65%, for example, approximately 51%, approximately 52%, approximately 53%, approximately 54%, approximately 55%, approximately 56%, approximately 57%, approximately 58%, approximately 59%, approximately 60%, approximately 61%, approximately 62%, approximately 63%, approximately 64%, or approximately 65% of the weight of the extract.

A β-glucan-rich Saccharomyces cerevisiae yeast cell wall extract according to the present invention also contains mannans in a total amount less than 5% by weight. The mannans derived from yeast cell walls are referred to as “cell wall mannans”. They are yeast polysaccharides constituted predominantly of mannose and more precisely of copolymers of neutral or acidic oses (with 5 or 6 carbon atoms), which are linked together by glycosidic bonds and bound to proteins. In Saccharomyces cerevisiae cell wall mannans, mannose is present therein in the form of a backbone of α-(1,6)-linked mannose residues (50 or more), branched by short chains of α-(1,2)- and α-(1,3)-linked mannose. The person skilled in the art knows how to extract mannans from yeast cell walls. A common method includes enzymatic digestion of yeast cell walls with a preparation of β-glucanases (for example the industrial preparation GLUCANEX™), followed by separation of the hydrolysate by means of centrifugation, and purification by means of ultrafiltration. Another method is based on a hot chemical extraction.

As used herein, the term ‘total amount of less than 5% by weight of mannans’ indicates that the mannans represent at most 5% of the weight of the extract according to the invention. Thus, the total amount of mannans present in an extract according to the invention may represent between 0.5% and 5% of the weight of the extract, in particular between 0.5% and 1.5% of the weight of the extract.

Preferentially, a β-glucan-rich Saccharomyces cerevisiae yeast cell wall extract according to the present invention comprises β-glucans and mannans, wherein the β-glucans are present in the form of a mixture of β-1,3-glucans and β-1,6-glucans in a total amount greater than or equal to 50% by weight; and the mannans are present in a total amount such that the ratio of β-glucans/mannans is between 12 and 40 (weight/weight). In particular, the β-glucan/mannan ratio in an extract according to the invention may be between 15 and 30 (weight/weight), and more particularly between 18 and 22 (weight/weight).

In certain embodiments, a β-glucan-rich Saccharomyces cerevisiae yeast cell wall extract according to the present invention further comprises a protein content less than or equal to 10% by weight, and a glycogen content less than or equal to 10% by weight.

In certain embodiments, a β-glucan-rich Saccharomyces cerevisiae yeast cell wall extract according to the present invention comprises, as the only active ingredients, β-glucans and mannans, wherein the β-glucans are present in the form of a mixture of β-1,3-glucans and β-1,6-glucans in a total amount greater than or equal to 50% by weight; and the mannans are present in a total amount less than 5% by weight such that the β-glucan/mannan ratio is between 12 and 40 (weight/weight), in particular between 15 and 30 (weight/weight), and more particularly between 18 and 22 (weight/weight).

In certain particular embodiments, a β-glucan-rich Saccharomyces cerevisiae yeast cell wall extract according to the invention has a dry matter content greater than or equal to 94% by weight, preferably greater than or equal to 96% by weight. A dry matter content greater than or equal to 94%, preferably 96% by weight, enables better conservation of the yeast hulls, in particular enhanced bacteriological stability and enhanced stability against undesirable reactions of enzymatic or non-enzymatic origin.

In certain particular embodiments, a Saccharomyces cerevisiae yeast cell wall extract according to the invention is in powder form.

In certain particular embodiments, the β-glucan-rich Saccharomyces cerevisiae yeast cell wall extract according to the invention is Safglucan® which is produced by the Applicant, Lesaffre. Safglucan® comprises β-glucans and mannans, as the only active ingredients, wherein the β-glucans are present in the form of a mixture of β-1,3-glucans and β-1,6-glucans in a total amount of between 52 and 60% by weight; and the mannans are present in a total amount of between 1 and 5% by weight. Safglucan® further also comprises a protein content less than or equal to 10% by weight, and a glycogen content less than or equal to 10% by weight.

After manufacture, a β-glucan-rich Saccharomyces cerevisiae yeast cell wall extract according to the invention may be packaged and/or stored under appropriate conditions, generally in a dry and cool place, prior to use. The shelf life of a cell wall extract according to the invention, in appropriate packaging, is 2 years as from the date of production.

### II—Compositions Comprising a β-Glucan-Rich Saccharomyces cerevisiae Yeast Cell Wall Extract

In certain embodiments, the β-glucan-rich Saccharomyces cerevisiae yeast cell wall extracts described herein are used as such, in particular without any other active substance present.

In other embodiments, the β-glucan-rich Saccharomyces cerevisiae yeast cell wall extracts described herein are used in combination with at least one other bioactive agent. Consequently, the present invention relates to a composition comprising a β-glucan-rich Saccharomyces cerevisiae yeast cell wall extract, as described herein, combined with at least one bioactive agent.

As indicated herein, the term “bioactive agent” refers to an agent that is beneficial for animal health. It may be a veterinary active substance, having a therapeutic or preventive effect. The bioactive agent may, for example, be selected from among antibiotics, vaccines, probiotics or prebiotics, or mixtures thereof.

In certain embodiments, the bioactive agent is selected from among antibiotics, vaccines or prebiotics.

In particular, it does not include a probiotic, in particular a microbial pesticide such as a strain QST-713 of Bacillus subtilis.

By extension the term ‘bioactive agent’ also refers to substances that have a beneficial effect from a nutritional standpoint, in particular nutrients such as, for example, vitamins, minerals, or trace elements, in particular those generally found in supplements, adjuvants or feed additives for animals.

The person skilled in the art knows how to select the components that are appropriate depending on the animal for which the composition is intended, in this instance appropriate for pigs, and knows how to determine the appropriate quantities to be included in such a composition.

The compositions according to the invention may be in any form, for example in the form of a powder, granules, a gel or a liquid. Preferably, the compositions according to the invention are in solid form, preferably in powder form.

The invention also relates to a pharmaceutical composition comprising a β-glucan-rich Saccharomyces cerevisiae yeast cell wall extract, as described herein, and at least one physiologically acceptable excipient for use in veterinary medicine. In the pharmaceutical composition, the extract may be present as the sole active ingredient. Alternatively, the pharmaceutical composition may additionally comprise at least one other bioactive agent, such as those listed above.

A pharmaceutical composition according to the invention may be classified as a veterinary pharmaceutical preparation available by prescription or over the counter.

In the context of the present invention the term ‘physiologically acceptable excipient for use in veterinary medicine’ refers to any medium or additive which does not interfere with the efficacy of the biological activity of the active ingredient (in this instance β-glucan-rich cell wall extract), and which is not excessively toxic for the animal, at the concentrations in which it is administered.

The veterinary pharmaceutical compositions according to the present invention may be administered using any combination of dosage and route of administration that is effective in achieving the desired prophylactic effect. Preferably, the pharmaceutical compositions are administered via the oral route. The person skilled in the art will recognise that the exact amount to be administered to pigs may vary according to the weight of the animal, and the effects of Lawsonia intracellularis on the animal to be treated.

### III—Uses of Dry β-Glucan-Rich Saccharomyces cerevisiae Yeast Cell Wall Extracts

As indicated above, Lawsonia intracellularis infections in pigs lead to inflammation of the intestine, and in particular ileitis, which causes diarrhoea and in some cases even sudden death. As a result of these infections, the animal experiences a drop in both feed efficiency and weight gain.

The β-glucan-rich Saccharomyces cerevisiae yeast cell wall extracts described herein have been shown to be capable of significantly reducing the symptoms associated with a Lawsonia intracellularis infection.

The invention therefore also relates to a β-glucan-rich Saccharomyces cerevisiae yeast cell wall extract as described herein, taken into consideration alone or in combination with at least one bioactive agent, for use in the prevention or treatment of Lawsonia intracellularis infection in pigs.

The invention therefore also relates to a β-glucan-rich Saccharomyces cerevisiae yeast cell wall extract, as described herein, taken into consideration alone or in combination with at least one bioactive agent, for use in the prevention or treatment of a disease associated with or caused by a Lawsonia intracellularis infection in pigs.

The disease associated with or caused by Lawsonia intracellularis may be an intestinal inflammation, in particular selected from the group consisting of enteritis or ileitis.

The invention also relates to a β-glucan-rich Saccharomyces cerevisiae yeast cell wall extract, as described here, taken into consideration alone or in combination with at least one bioactive agent, for use in the prevention or treatment of inflammatory intestinal diseases in pigs, in particular associated with or caused by a Lawsonia intracellularis infection, such as ileitis.

As used here, the term “prevention” refers to any act aimed at preventing or delaying the onset of a disease, in particular reducing or inhibiting, partially or totally, the probability of a subject developing one or more symptoms linked to a disease associated with or caused by a Lawsonia intracellularis infection (diarrhoea, reduction in body weight, thickening of the intestinal wall, etc). The terms “prevention” and “prophylactic treatment” are equivalent and may be used interchangeably in the context of the present invention. Prevention may be targeted at subjects having a predisposition to infections, preferably intestinal infections, as well as subjects who are not so predisposed.

As used herein, the term ‘treatment’ refers to any act aimed at improving the medical status of a subject suffering from a disease. The treatment may be aimed at improving the patient's medical condition, i.e. regressing the disease or certain symptoms thereof, or eradicating the disease or certain symptoms thereof. The treatment may also have the effect of preventing or slowing down the progression of a disease.

In some embodiments of the present invention, the extracts or compositions are intended for use in the prevention of a disease associated with or caused by Lawsonia intracellularis in pigs.

In certain embodiments of the present invention, the use of a β-glucan-rich Saccharomyces cerevisiae yeast cell wall extract, alone or in combination with at least one bioactive agent, in the prevention of a disease associated with or caused by the bacterium Lawsonia intracellularis, comprises the admixing of a β-glucan-rich Saccharomyces cerevisiae yeast cell wall extract with an animal feedstuff. The admixing may be done by any appropriate method known in the art.

As used herein, the terms “animal feed”, “animal feed ingredient” and “animal feed product” refer to any natural or processed organic material that is readily biodegradable and is suitable for consumption by an animal. Examples of such organic materials range from freshly harvested grains to pelleted feedstuffs, and include, for example, any compound, grain, nut, fodder, silage, preparation, composition or mixture suitable or intended for ingestion by an animal. The animal feedstuff may be in the form of pellets, liquid, slurry, or so-called “mash feed”.

The term ‘fodder’ refers to plant material (mainly plant leaves and stems) consumed by grazing animals. The term ‘silage’ refers to fermented fodder having a high moisture content that is suitable for use as feed for ruminants. Preparations, compositions and admixtures, for example commercial preparations, compositions and admixtures, may be in any appropriate form, for example in the form of concentrates, premixes, supplements, total mixed ration (TMR), etc. The animal feed may contain cereals (e.g. maize, wheat, barley, rye, rice, sorghum, millet, or a combination thereof), fodder, silage, pulses (e.g. soya), spent grain, animal products, etc. The spent grain is mainly derived from the processing of maize, wheat, barley, rye, rice, sorghum, millet or a combination thereof. Spent grains are mainly generated as a result of industrial processes such as brewing and distilling, which are geared towards the production of potable alcohol (whisky, vodka or gin) or biofuels. Brewers' grains are generated from malt, which in turn is derived from barley. Cereals other than barley may occasionally be used in brewing, such as sorghum, particularly in Africa. The spent grains resulting from bioethanol production are derived from maize or wheat. Wet spent grains are dried in order to produce the dry spent grains, which are used primarily as animal feed.

A β-glucan-rich Saccharomyces cerevisiae yeast cell wall extract, as described herein, taken into consideration alone or in combination with at least one bioactive agent may be added to an animal feedstuff in amounts of approximately 0.02% to approximately 0.05% by total weight of feedstuff (which corresponds to amounts of approximately 200 g to approximately 500 g per tonne of feedstuff) subject to compliance with the applicable current legislation. For example, in certain embodiments, a β-glucan-rich Saccharomyces cerevisiae yeast cell wall extract, as described herein, taken into consideration alone or in combination with at least one bioactive agent, is added to an animal feedstuff in an amount of approximately 0.02% to approximately 0.05% by total weight of the feedstuff (which corresponds to an amount of approximately 200 to approximately 500 g per tonne of feedstuff), for example in an amount of approximately 0.02% to approximately 0.05% by weight of the feedstuff (which corresponds to approximately 200 to approximately 500 g per tonne of feedstuff).

The extract or compositions according to the invention may be administered throughout the entire duration of the infection or the entire duration of the risk of infection. In particular, it may be administered continuously, together with the feed, with a view to preventing: diseases associated with or caused by the bacterium Lawsonia intracellularis; and/or Lawsonia intracellularis infections.

Unless otherwise defined, all technical and scientific terms used in this Description have the same significance/meaning as that commonly understood by an ordinary person skilled in the art who is a specialist in the field to which this invention belongs.

### Examples

The following examples describe certain embodiments of the present invention. However, it is understood that the examples and figures are presented for purely illustrative purposes only and in no way limit the scope of the invention.

### Materials and Methods

160 piglets (Danbred×Duroc) were obtained from a commercial farm and placed in a fattening/finisher unit in which Lawsonia intracellularis bacteria were present. On arrival at the farm, they were individually weighed and ear-tagged. The animals were then divided into two groups (1 control group and 1 treated group) of 80 pigs each. The animals were then separated into sub-groups of 10 animals each. The control group received the standard feed. The treated group received the normal feed in which the beta glucan product (250 ppm) was included throughout the entire duration of the trial. The animals in the 2 groups were weighed on days 63, 81 and 97; while their faeces were collected at the same time in order to test for the presence of Lawsonia intracellularis bacteria by means of quantitative qPCR.

### Results

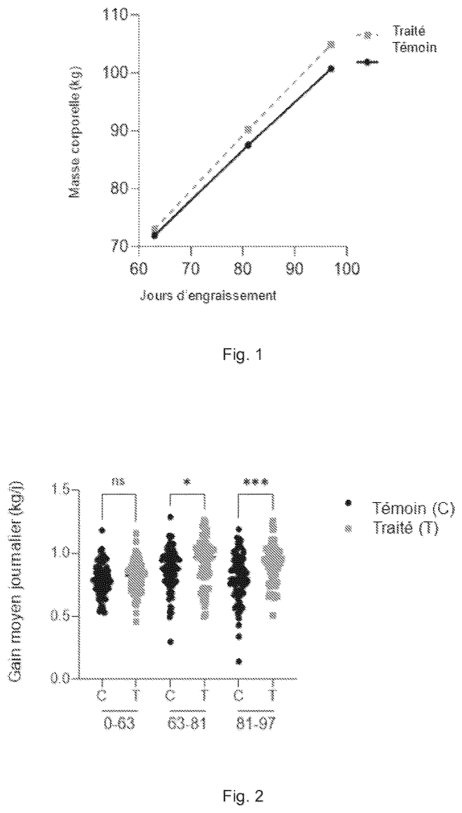

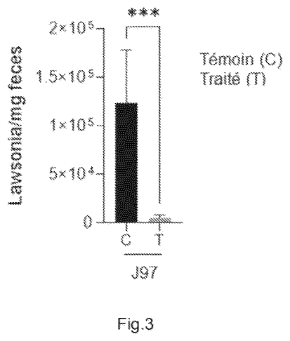

The results show that after 97 days of treatment, the treated animals each gained around 5 kg on average as compared to the control group (FIGS. 1 and 2),
The presence of Lawsonia intracellularis in the faeces was drastically reduced (FIG. 3).

1. A method for the prevention or treatment of a disease associated with or caused by a Lawsonia intracellularis infection in a pig comprising administering to the pig a β-glucan-rich Saccharomyces cerevisiae yeast cell wall extract, the extract comprising β-glucans and mannans,
wherein the β-glucans are present in the form of a mixture of β-1,3-glucans and β-1,6-glucans in a total amount greater than or equal to 50% by weight, notably between 50% and 99% by weight; and
wherein the mannans are present in a total amount less than 5% by weight, notably between 1% and 5% by weight.

2. The method of claim 1, wherein the disease is an inflammatory intestinal disease, in particular ileitis.

3. The method of claim 1, wherein the total amount of β-glucans present in a β-glucan-rich Saccharomyces cerevisiae yeast cell wall extract is between 50% and 90% of the weight of the extract, for example between 50% and 80%, or between 50% and 70%, or indeed between 50% and 60%, of the weight of the extract.

4. The method of claim 1, wherein the total amount of mannans present in a β-glucan-rich Saccharomyces cerevisiae yeast cell wall extract is between 2% and 4% of the weight of the extract.

5. The method of claim 1, wherein the amount of mannans in the β-glucan-rich Saccharomyces cerevisiae yeast cell wall extract is such that the ratio of β-glucans/mannans is between 12 and 40 (weight/weight).

6. The method of claim 1, wherein the amount of mannans in the β-glucan-rich Saccharomyces cerevisiae yeast cell wall extract is such that the ratio of β-glucans/mannans is between 15 and 30 (weight/weight), in particular between 18 and 22 (weight/weight).

7. The method of claim 1, wherein the extract has a dry matter content greater than or equal to 94% by weight, in particular greater than or equal to 96% by weight.

8. The method of claim 1, wherein the extract further comprises a protein content less than or equal to 10% by weight, and a glycogen content less than or equal to 10% by weight.

9. The method of claim 1, wherein the β-glucans and mannans are the only active ingredients of the β-glucan-rich Saccharomyces cerevisiae yeast cell wall extract.

10. The method of claim 9, wherein the extract is Safglucan® which comprises β-glucans and mannans, wherein the β-glucans are present in the form of a mixture of β-1,3-glucans and β-1,6-glucans in a total amount of between 52 and 60% by weight; and the mannans are present in a total amount of between 1 and 5% by weight; and wherein the Safglucan® further comprises a protein content less than or equal to 10% by weight, and a glycogen content less than or equal to 10% by weight.

11. A method for the prevention of an infection associated with the bacterium Lawsonia intracellularis in a pig comprising: administrating a composition comprising a β-glucan-rich Saccharomyces cerevisiae yeast cell wall extract and at least one bioactive agent the extract comprising β-glucans and mannans,
wherein the β-glucans are present in the form of a mixture of β-1,3-glucans and β-1,6-glucans in a total amount greater than or equal to 50% by weight, notably between 50% and 99% by weight; and
wherein the mannans are present in a total amount less than 5% by weight, notably between 1% and 5% by weight.

12. The method of claim 11, wherein the bioactive agent is selected from the group consisting of antibiotics, vaccines, probiotics, prebiotics, vitamins, minerals, trace elements, and mixtures thereof.

13. A method for the prevention of an infection associated with the bacterium Lawsonia intracellularis in a pig comprising: administrating a pharmaceutical composition comprising a β-glucan-rich Saccharomyces cerevisiae yeast cell wall extract and at least one physiologically acceptable excipient the extract comprising β-glucans and mannans,
wherein the β-glucans are present in the form of a mixture of β-1,3-glucans and β-1,6-glucans in a total amount greater than or equal to 50% by weight, notably between 50% and 99% by weight; and
wherein the mannans are present in a total amount less than 5% by weight. notably between 1% and 5% by weight.

14. (canceled)

15. The method of claim 1, wherein the administering comprises feeding.

16. The method of claim 11, wherein the administering comprises feeding.
